(19) United States
(12) Patent Application Publication
(10) Pub. No.: US 2026/0123823 A1
(43) Pub. Date: May 7, 2026

(54) PACKAGING FOR A MEDICAL ENDOSCOPE

(71) Applicant: AXESS VISION TECHNOLOGY, JOUE-LES-TOURS (FR)

(72) Inventors: Emmanuel HALLAUER, SACHE (FR); Philippe LE ROUX, TOURS (FR); Luka LECOUTEY, ROCHECORBON (FR); Jéröme COURATIN, SAINT-PIERRE-DES-CORPS (FR)

(21) Appl. No.: 19/116,390

(22) PCT Filed: Sep. 28, 2023
(86) PCT No.: PCT/FR2023/051492

§ 371 Date: Mar. 28, 2025

(30) Foreign Application Priority Data
Sep. 29, 2022 (FR) FR2209870

(51) Int. Cl. A61B 1/00

(52) U.S. Cl. CPC A61B 1/00144

(57) ABSTRACT

A packaging for a medical endoscope is disclosed. The packaging comprises a tray provided with an inner face having a peripheral area. The tray comprises, on its inner face, a housing for receiving the medical endoscope. The housing includes a cavity that receives the operating handle and a rectilinear groove that receives the insertion tube. The packaging additionally comprises a sheet sealed continuously and hermetically to the peripheral area of the inner face of the tray with a pull-out force. The tray comprises protuberances projecting from its inner face and arranged on either side of the groove. The sheet is fixed to the protuberances with a pull-out force at least 30% less than the pull-out force between the sheet and the peripheral area. The present disclosure also relates to a method for packaging a medical endoscope.

### TECHNICAL FIELD

The present disclosure relates to a packaging for a medical endoscope, of a reusable or single-use nature.

Medical endoscopes allow access to the inner surface of a hollow organ, cavity or natural or artificial conduit of the human body in order to perform various operations for therapeutic, surgical or diagnostic purposes. Medical endoscopes are used for the inspection of all inner parts of the human body accessible by natural or artificial means. For example, medical endoscopes can be used in the field of urinary tract, gastrointestinal tract, respiratory system, cardiovascular system, trachea, sinus cavity, female reproductive system, abdominal cavity or any other part of the human body to be explored by a natural or artificial means.

Given their use, medical endoscopes are stored in sterile packaging, which constitutes a sterile barrier and protects them from possible damage that could result from shocks during transport in particular.

### BACKGROUND

A medical endoscope comprises an operating handle, an insertion tube and a power cable. It is usually packaged in a tray or tray covered with plastic film. The tray comprises a housing for receiving the medical endoscope, one part of which accommodates the operating handle, which is an extension of a second part for receiving the insertion tube and possibly a third part for receiving the power cable. This tray is covered with a plastic film allowing sterilization and constituting a sterile barrier. In general, the film is hermetically fixed around the edge of the tray to ensure the sterility of the medical endoscope.

Conventionally, the housing comprises a rib in the shape of an arc of a circle or a circular shape to receive the insertion tube, as is apparent from patents U.S. Pat. No. 5,947,284 or EP 3 148 396 in particular. However, the insertion tube can become deformed and damaged when stored in this position, and it is preferable to have a packaging in which the tube is held in a rectilinear position in line with the operating handle.

To keep the medical endoscope in position in the packaging, the packaging may comprise clips, for example. However, inserting or removing the medical endoscope from its housing can be difficult, and when the clips are located on the insertion tube, the latter can be damaged during these insertion and removal operations. To solve this problem, various solutions have been considered. For example, patent U.S. Pat. No. 5,947,284 proposes inserting the insertion tube of the medical endoscope into an arc-shaped tube which then serves as a guide for removing the insertion tube. However, this solution has the disadvantage of creating a lot of vibration and stress, particularly during transport. In addition, this solution can be difficult to implement for the practitioner, not only to remove the medical endoscope from its packaging, but also if he wishes to place it during use. In addition, this solution is not economical since it requires the preparation of a tube adapted to the packaging. Finally, the insertion tube is stored in an arc-shaped position, which has the risks mentioned above.

This same patent U.S. Pat. No. 5,947,284 proposes another solution for holding the medical endoscope in position in the packaging, namely the use of a second film to cover the part of the housing containing the insertion tube. For this purpose, fixing points for this second film are arranged around the edge of the tray, close to the peripheral area. This solution has the disadvantage of not holding the insertion tube sufficiently well in position in the packaging. In addition, this solution can be difficult to implement for the practitioner because of the two films that must be removed to remove the endoscope from its packaging. Here again, the insertion tube is placed in a housing in the shape of an arc of a circle, which has the disadvantages mentioned above.

There is therefore a need for a packaging for a medical endoscope that is sterilizable and sufficiently robust to constitute a sterile barrier until its use. There is also a need for a packaging that is easy to use: it is advantageously easy to open, and preferably allows the practitioner to easily lay the medical endoscope during use so that it can be reused later. There is also a need for a packaging that can securely hold the medical endoscope in position: it is preferable that the operating handle and the insertion tube are correctly held in position in their housing so that they are not damaged during storage or transport. There is also a need for a packaging that can keep the insertion tube in a straight position, and in line with the operating handle.

### SUMMARY

The present disclosure aims at overcoming the disadvantages of the prior art by proposing a packaging of a medical endoscope comprising an operating handle and an insertion tube, said packaging comprising:
a tray provided with an inner face having a peripheral area, said tray comprising on its inner face a housing receiving the medical endoscope, the housing comprising a cavity receiving the operating handle, and a rectilinear groove receiving the insertion tube, the groove having a section greater than the section of the insertion tube to allow its free insertion,
a sheet permeable to gases and beta and gamma radiation, impermeable to microbes, sealed continuously and hermetically to the peripheral area of the inner face of the tray with a pull-out force, characterized in that the tray comprises protuberances projecting from its inner face and arranged on either side of the groove, and in that the sheet is fixed to the protuberances with a pull-out force at least 30% less than the pull-out force between the sheet and the peripheral area.

In the context of the present disclosure, the pull-out force between the sheet and the peripheral area (or protuberances) corresponds to the force required per unit area to pull the sheet from the peripheral area (or protuberances). The pull-out force can be measured in N/mm².

In the context of the present disclosure, a “pull-out force between the sheet and the protuberances at least 30% less than the pull-out force between the sheet and the peripheral area” means that the ratio between the pull-out force between the sheet and the protuberances and the pull-out force between the sheet and the peripheral area at isosurface (that is to say for the same contact surface between the sheet and the protuberances on the one hand, and between the sheet and the peripheral area on the other hand) is less than or equal to 0.7.

The packaging according to the present disclosure allows to keep the insertion tube in position straight and in line with the operating handle, and thus to ensure better preservation of the insertion tube.

The packaging according to the present disclosure allows to ensure a sterile barrier for the medical endoscope, thanks to the continuous and hermetic sealing between the tray and the sheet which is impermeable to microbes.

The presence of protuberances on either side of the groove allows the insertion tube to be held in position in the packaging, without deformation or crushing.

The packaging according to the present disclosure is easy to use, both for placing the endoscope in its packaging and for opening the packaging by the practitioner. Opening is easy and quick. According to an advantageous embodiment of the present disclosure, the practitioner can replace the medical endoscope in its packaging during his intervention if necessary, and use it again afterwards for the same patient and during the same intervention for example.

The packaging according to the present disclosure advantageously has either one of the following features, or a combination thereof:
the protuberances are positioned at the border of the groove;
the protuberances are distributed along the groove;
the protuberances are arranged alternately on one side or the other of the groove;
the housing comprises a gutter receiving a power cable of the medical endoscope;
at least one protuberance is positioned at the border of the gutter;
at least one protuberance is present in the gutter in order to wind the power cable;
the protuberances are studs or ribs;
the packaging comprises two stop lines arranged in the parts of the peripheral area located on either side of the groove;
the housing includes clips for holding the operating handle in position;
the housing comprises a cell receiving a connector of the power cable of the medical endoscope;
the packaging comprises a hanging slot in the peripheral area;
the packaging further comprises an adhesive surface on the inner face of the tray which is covered with a peelable film.

The present disclosure also relates to a method for packaging a medical endoscope comprising the following steps:
a) having a medical endoscope including an operating handle, an insertion tube, and optionally a power cable,
b) having a tray provided with an inner face having a peripheral area, said tray comprising on its inner face a housing intended to receive a medical endoscope, the housing being composed of a cavity intended to receive the operating handle, a rectilinear groove intended to receive the insertion tube, and optionally a gutter intended to receive the power cable, the groove having a section greater than the section of the insertion tube to allow its free insertion, said tray comprising protuberances projecting from its inner face and arranged on either side of the groove,
c) providing a sheet permeable to gases and beta and gamma radiation, impermeable to microbes and heat-sealable,
d) placing the medical endoscope in the housing,
e) contacting the heat-sealable sheet with the peripheral area and with the protuberances, heating to a sealing temperature, applying a peripheral sealing pressure on the peripheral area allowing a hermetic sealing of the sheet on the peripheral area to obtain a pull-out force, and applying a central sealing pressure on the protuberances allowing the sheet to be fixed on the protuberances to obtain a pull-out force, the peripheral sealing pressure and the central sealing pressure being such that the pull-out force between the sheet and the protuberances is at least 30% less than the pull-out force between the sheet and the peripheral area.

Advantageously, step b) of the method according to the present disclosure is such that either the peripheral area and the protuberances project at the same height relative to the inner surface of the tray, or the protuberances project at a height less than that of the peripheral area relative to the inner surface of the tray.

Finally, the present disclosure relates to a packaging of a medical endoscope obtained in accordance with the method according to the present disclosure.

### BRIEF DESCRIPTION OF THE DRAWINGS

FIG. 1 is a top view showing a tray of a packaging for receiving an endoscope in accordance with a first variant embodiment of the present disclosure in which the protuberances are in the form of studs.

FIG. 2 is a top view showing a medical endoscope in a packaging according to a first embodiment of the present disclosure according to which the protuberances are in the form of studs.

FIG. 3 is a sectional view along line BB of FIG. 2 showing the insertion tube of a medical endoscope in the groove of a packaging in accordance with the present disclosure.

FIG. 4 shows a detailed top view showing a packaging in accordance with a second variant embodiment of the present disclosure according to which the protuberances are in the form of ribs and studs.

FIG. 5 is a top view showing a medical endoscope in a packaging in accordance with the second variant embodiment of the present disclosure in which the protuberances are in the form of studs and ribs.

FIG. 6 is a sectional view taken along lines AA of FIG. 5.

FIG. 7 is a sectional view taken along lines II of FIG. 5.

FIG. 8 is a top view showing a medical endoscope in a packaging, further comprising a cell for the cable connector.

FIG. 9 is a top view showing a medical endoscope in a packaging, comprising an adhesive surface for resealing the packaging after opening.

### DESCRIPTION OF THE EMBODIMENTS

As shown in the figures, the present disclosure relates to a packaging 1 for a medical endoscope 2, conventionally composed of an operating handle 3, an insertion tube 4 and optionally a power cable 5. The packaging 1 according to the present disclosure includes a tray 6 arranged to receive the medical endoscope 2 and closed by a sheet 7 assembled with the tray 6.

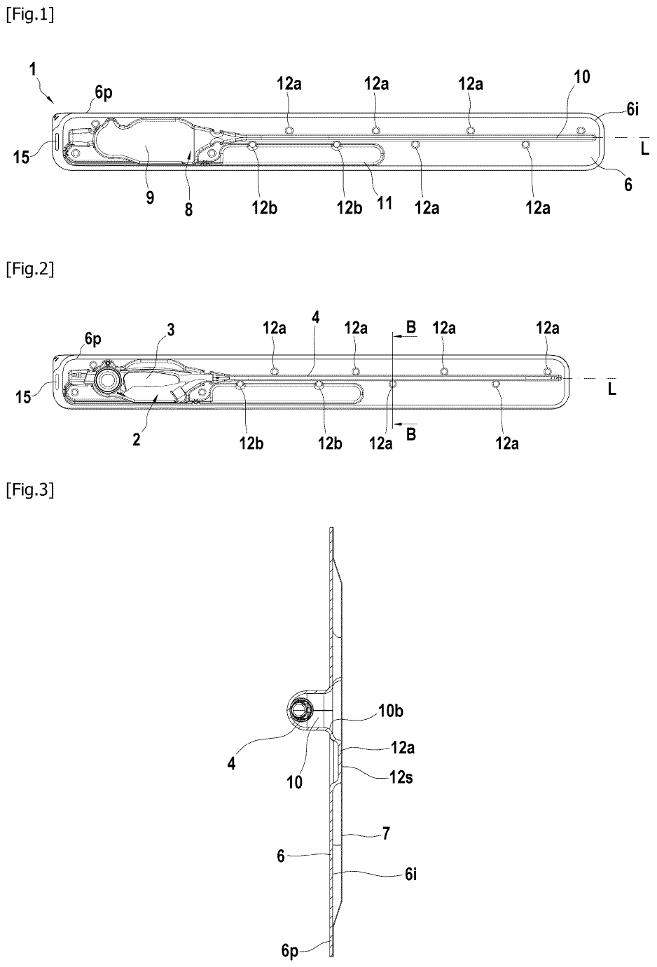

As shown in FIG. 1, the tray 6 is provided with an inner face 6ᵢ having a peripheral area 6ₚ. According to a first embodiment illustrated in FIGS. 2 and 3, the peripheral area 6ₚ does not protrude from the inner face 6ᵢ of the tray 6 but is flush with the inner face 6ᵢ, that is to say that the peripheral area 6ₚ and the inner face 6ᵢ of the tray 6 belong to the same plane. Alternatively, according to the embodiment illustrated in FIGS. 5 to 7, the peripheral area 6ₚ projects from the inner face 6ᵢ of the tray 6, thus forming a peripheral rim.

The tray 6 includes a housing 8 on its inner face 6ᵢ in order to receive the medical endoscope 2, as illustrated in FIG. 1. This housing 8 is composed of a cavity 9 opening into a rectilinear groove 10, as well as into a gutter 11. Although not illustrated, the housing 8 may not have a gutter 11 in the case where the medical endoscope 2 does not have a power cable 5.

The cavity 9 is intended to receive the operating handle 3. Its dimensions allow the operating handle 3 to be fully inserted so that it does not protrude from the inner face 6ᵢ of the tray 6. According to a particular embodiment of the present disclosure, the cavity 9 comprises at least one clip for holding the operating handle 3 in position.

The gutter 11 is intended to receive the power cable 5. The dimensions and shape of the gutter 11 are such that the entire power cable 5 can be inserted therein, so that it does not protrude from the inner surface 6ᵢ of the tray 6.

According to an embodiment illustrated in FIG. 8, the power cable 5 is provided at its free end with a connector 5_{c}. According to this embodiment, the housing 8 further comprises a cell 11ₐ intended to receive the connector 5_{c}. More precisely, the gutter 11 opens into the cell 11ₐ. The cell 11ₐ may include at least one clip to hold the connector 5_{c} in position. According to this embodiment, the connector 5_{c} does not protrude from the inner surface 6ᵢ of the tray 6.

The groove 10 is intended to receive the insertion tube 4 of the medical endoscope 2. The groove 10 therefore has a section greater than the section of the insertion tube 2 to allow its free insertion. Advantageously, the section of the groove 10 is greater than the section of the insertion tube with an insertion clearance allowing free engagement without jamming of the insertion tube 4 in the groove 10. Thus the insertion tube 4 is held laterally by the walls of the groove 10 while being able to be inserted or removed without blocking. The risks of damage to the insertion tube 4 during assembly and removal operations are thus avoided. The groove 10 is rectilinear along a longitudinal axis L, so that the insertion tube 4 is also rectilinear when it is placed in the packaging 1. Preferably, the groove 10 is tubular in shape. The groove 10 opens on the inner face 6ᵢ by its edge 10_{b}, as illustrated in FIG. 3.

The tray 6 also comprises at least two protuberances having an upper face 12ₛ and projecting from the inner face 6ᵢ of the tray 6.

In the context of the present disclosure, the tray 6 comprises at least two protuberances 12ₐ arranged on either side of the groove 10, that is to say on each side of the groove 10. In other words, these protuberances 12ₐ project from the inner face 6ᵢ of the tray 6, between the peripheral area 6ₚ and the housing 8. Preferably, these protuberances 12ₐ are positioned at the border of the groove 10, that is to say that the protuberances 12ₐ are contiguous to the edge 10_{b} of the groove. Advantageously, the protuberances 12ₐ are contiguous to the groove 10, and in particular the distance between each protuberance 12ₐ and the edge 10_{b} of the groove 10 is less than the diameter of the insertion tube 4, which allows to prevent the insertion tube 4 from coming out of the groove 10. These protuberances 12ₐ are advantageously distributed from one end of the groove 10 to the other, although this is not obligatory and they could be arranged along a part of the groove 10. Preferably, the protuberances 12ₐ are arranged alternately on one side or the other of the groove 10. Although not preferred, it could be considered that the protuberances 12ₐ are arranged opposite each other on either side of the groove 10. Preferably, the protuberances 12ₐ are distributed at regular intervals along the groove 10. According to one embodiment, the protuberances 12ₐ are aligned along two axes parallel to the longitudinal axis L of the groove 10, these two axes being at equal or unequal distance from the longitudinal axis L of the groove 10.

According to a particular embodiment of the present disclosure illustrated in FIG. 2 in particular, the tray 6 further comprises at least one protuberance 12_{b} positioned at the border of the gutter 11 in order to hold the power cable 5 in position therein. These protuberances 12_{b} may be located on either side of the gutter 11. Alternatively, as shown in FIGS. 1 and 2 in particular, the gutter 11 is arranged to pass through at least one protuberance 12_{b} so that the at least one protuberance 12_{b} extends on either side of the gutter 11. Thus, the same protuberance 12_{b} may be positioned to be at the border of both the groove 10 and the gutter 11.

According to a particular embodiment of the present disclosure illustrated in FIG. 5 in particular, the tray 6 further comprises one or more protuberances 12_{c} positioned in the gutter 11 in order to hold the power cable 5 in position in the gutter 11. Thus according to this embodiment, the power cable 5 is wound around the protuberances 12_{c} which are mounted in the gutter 11.

According to a first embodiment, the protuberances 12ₐ, 12_{b}, 12_{c} may be discrete elements, and are then in the form of studs or columns, as shown in FIGS. 1 to 3 in particular. Advantageously, the different studs bordering the groove 10 and/or the gutter 11 are of identical dimensions, although a slight variation is possible. According to this embodiment, the protuberances 12_{c} mounted in the gutter 11 may advantageously be of different dimensions from the protuberances 12ₐ bordering the groove 10 and the protuberances 12_{b} bordering the gutter 11, although this is not obligatory.

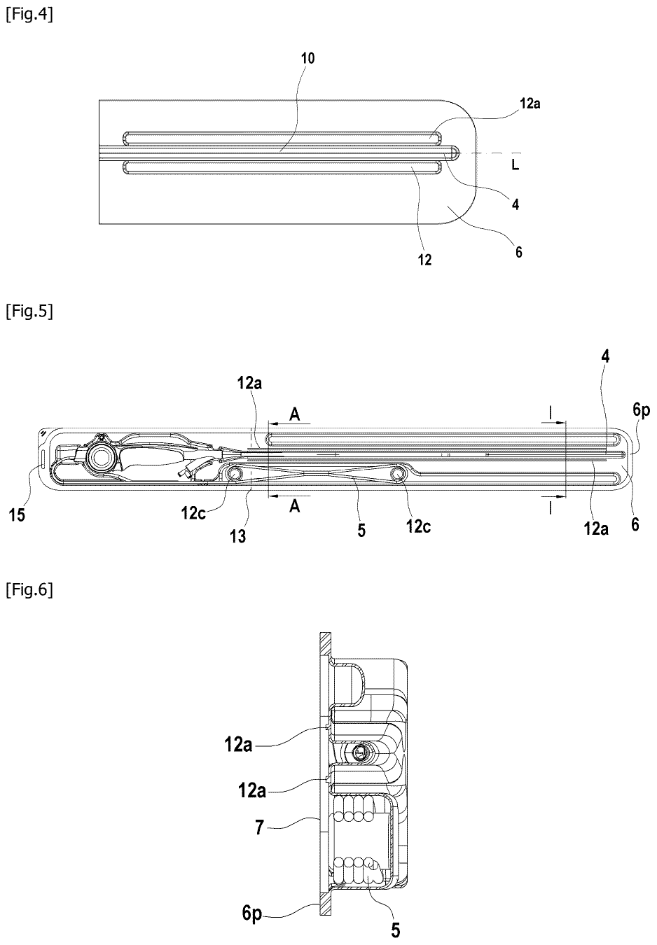

According to a second embodiment, the protuberances 12ₐ, 12_{b}, 12_{c} are in the form of ribs, preferably rectilinear and parallel to the longitudinal axis L of the groove 10, as shown in FIGS. 4 and 5. The dimensions of the ribs may be different or, preferably, equal. According to this embodiment, the groove 10 and/or the gutter 11 may be bordered by two ribs going from one of its ends to the other, that is to say that each of the two ribs has a length identical to the edge 10b of the groove 10 and/or to the edge of the gutter 11. Alternatively, the groove 10 and/or the gutter 11 may be bordered by at least two ribs of a length less than the length of the groove 10 and/or the gutter 11.

According to a third embodiment, certain protuberances 12ₐ, 12_{b}, 12_{c} are in the form of studs, and certain protuberances 12ₐ, 12_{b}, 12_{c} are in the form of ribs parallel to the longitudinal axis L. This embodiment is in particular illustrated in FIGS. 5 and 8.

The tray 6 can be made of any material allowing not only its attachment with the sheet 7, but also allowing it to contribute to the sterility of the medical endoscope 2. The tray 6 is typically made of thermoplastic material, and for example selected from polyethylene terephthalate (PET), amorphous polyethylene terephthalate (APET), glycolized polyethylene terephthalate (PETg), polystyrene, and polyvinyl chloride (PVC).

The packaging 1 according to the present disclosure further comprises a sheet or film 7 fixed to the peripheral area 6ₚ of the tray 6 and on the protuberances 12ₐ, 12_{b}, 12_{c}. This fixing can be carried out by heat-sealing for example.

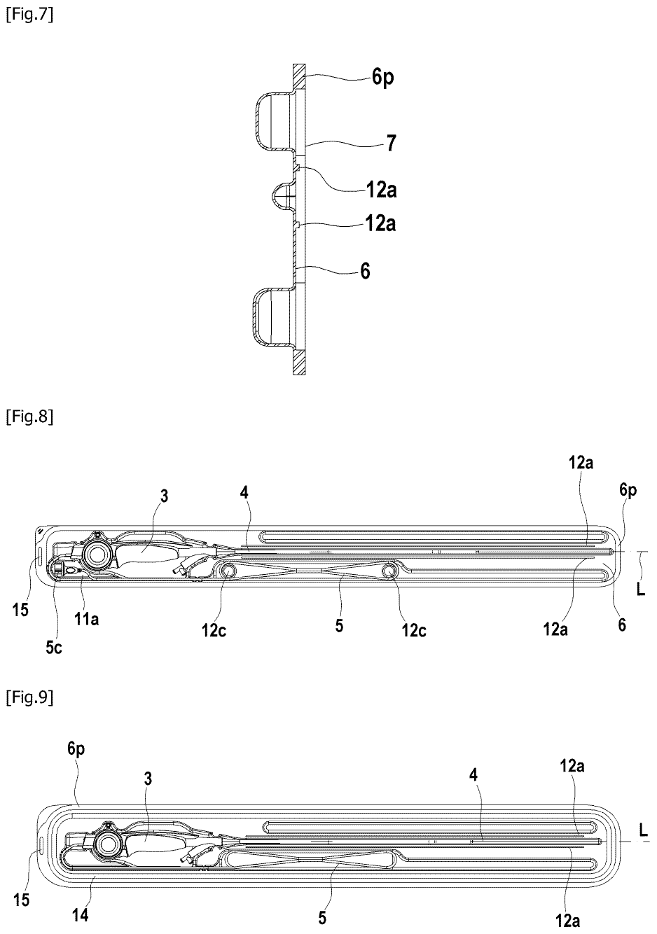

The peripheral area 6ₚ of the tray 6 and the sheet 7 are sealed. Thus, the attachment between the tray 6 and the sheet 7 is continuous and hermetic over the entire circumference of the inner face 6ᵢ of the tray 6. According to the embodiment illustrated in FIG. 3, the peripheral area 6ₚ does not protrude from the inner face 6ᵢ of the tray 6 and the sheet 7 is deformed in the direction of the tray. According to the embodiment illustrated in FIGS. 6 and 7, the peripheral area 6ₚ projects relative to the inner face 6ᵢ of the tray 6 and the sheet 7 is flat.

The attachment between the sheet 7 and the peripheral area 6ₚ of the tray 6 is defined by a pull-out force to pull the sheet 7 from the peripheral area 6ₚ of the tray 6. This pull-out force is such that the sheet can be removed from the tray without excessive force by the practitioner, while allowing effective and sterile closing of the packaging 1. Typically, the pull-out force between the sheet 7 and the peripheral area 6ₚ of the tray 6 ranges from 0.0015 N/mm² to 2 N/mm², preferably from 0.007 N/mm² to 0.03 N/mm². In the context of the present disclosure, the pull-out force can be measured according to standard EN 868-5.

Then, the sheet 7 is also fixed to the tray 6 via the protuberances 12ₐ, 12_{b}, 12_{c}. More precisely, the sheet 7 is fixed on the upper face 12ₛ of the protuberances 12ₐ, 12_{b}, 12_{c}, regardless of their shape. The fixing between the sheet 7 and the protuberances 12ₐ, 12_{b}, 12_{c} is defined by a pull-out force to pull the sheet 7 from the protuberances 12ₐ, 12_{b}, 12_{c}. The fixing of the sheet 7 on the protuberances 12ₐ bordering the groove 10 allows the insertion tube 4 to be held inside the groove 10, and the fixing of the sheet 7 on the protuberances 12_{b} bordering the gutter 11 allows the power cable 5 to be held inside the groove 11. Typically, the pull-out force between the sheet 7 and the protuberances 12ₐ, 12_{b}, 12_{c} ranges from 0.001 N/mm² to 0.1 N/mm², preferably from 0.005 N/mm² to 0.02 N/mm².

In the context of the present disclosure, the pull-out force between the sheet 7 and the protuberances 12ₐ, 12_{b}, 12_{c} is less than the pull-out force between the sheet 7 and the peripheral area 6ₚ of the tray 6. Thus, the opening of the packaging 1 by the practitioner is easy, while allowing to guarantee the sterility of the medical endoscope 2 which is inside the packaging 1 and its storage in a safe manner. More precisely, the pull-out force between the sheet 7 and the protuberances 12ₐ, 12_{b}, 12_{c} is at least 30% less than the pull-out force between the sheet and the peripheral area of the tray, that is to say the ratio between the pull-out force between the sheet 7 and the protuberances 12ₐ, 12_{b}, 12_{c} and the pull-out force between the sheet 7 and the peripheral area 6ₚ of the tray 6 at isosurface (that is to say for the same unit area) is less than or equal to 0.7. In addition, the number, shape, dimensions, and value of the pull-out force of the protuberances 12ₐ, 12_{b}, 12_{c} can be selected by the person skilled in the art according to the desired total pull-out forces and so as to allow the insertion tube 4 to be held inside the groove 10 and the power cable 5 to be held in the gutter 11.

The sheet 7 is made of any material allowing its attachment to the tray 6 and ensuring the sterility of the medical endoscope 2 by constituting a sterile barrier. Indeed, the sheet 7 must be permeable to beta and gamma radiation to allow the sterilization of the packaging 1 containing the medical endoscope 2. In addition, the sheet 7 must be impermeable to microbes to allow the medical endoscope 2 to be kept sterile in its packaging. For example, the sheet 7 can be made of plastic or a combination of paper and plastic. According to a preferred embodiment, the film is made of a plastic material, in particular thermoplastic, such as polyamide or polyethylene such as Tyvek® marketed by the company Dupont de Nemours for example.

According to a particular embodiment of the present disclosure shown in FIG. 5, the packaging 1 further comprises two stop lines 13 arranged in the peripheral area 6ₚ. These stop lines 13 are located on either side of the groove 10, and preferably belong to the same axis perpendicular to the longitudinal axis L of the groove 10. Thus, these stop lines cross the two sides of the peripheral area 6ₚ located on either side of the groove 10. Thus, when the practitioner opens the packaging 1, this opening (consisting of detaching the sheet 7 from the tray 6) can be made up to these two stop lines 13. The opening must then be large enough to allow the medical endoscope 2 to be easily removed from its packaging 1. Advantageously, the medical endoscope can be replaced in the packaging 1 easily by the practitioner, if necessary.

According to a particular embodiment, the packaging 1 further comprises an adhesive surface 14 on the inner face 6ᵢ of the tray 6, preferably facing the peripheral area 6ₚ as illustrated in FIG. 9, and/or covering part of the peripheral area 6ₚ. The adhesive surface 14 may be continuous or discontinuous. Preferably, the adhesive surface 14 is in the form of a contour going from one stop line 13 to the other, in the part of the inner face 6ᵢ of the tray 6 intended to be opened, that is to say on the side of the cavity 9. Alternatively, as illustrated in FIG. 9, this adhesive surface 14 is located around the entire periphery of the packaging, that is to say along the entire length of the peripheral area 6ₚ. The adhesive surface 14 is either contiguous of the peripheral area 6ₚ, or close to the peripheral area 6ₚ, and, regardless of the embodiment, possibly partially covering the peripheral area 6ₚ. The adhesive surface 14 is preferably positioned at the border of the cavity 9. Advantageously, the adhesive surface 14 is positioned between the peripheral area 6ₚ and the housing 8. According to this embodiment, the adhesive surface 14 is covered with a peelable film. Thus, according to this embodiment, the practitioner can open the packaging 1 completely or up to the two stop lines 13, use the medical endoscope 2 as needed, replace it in the packaging, remove the peelable film and close the packaging 1 by contacting the adhesive surface 14 with the sheet 7. Depending on the needs of the practitioner, the packaging can be opened or closed.

According to a particular embodiment, the packaging 1 according to the present disclosure comprises a hanging slot 15 in the peripheral area 6ₚ, in order to be able to suspend the packaging 1. Preferably, this slot 15 is oblong. Preferably, this slot 15 is located in the part of the peripheral area 6ₚ located opposite the cavity 9 intended to receive the operating handle 3.

The present disclosure also relates to a method for packaging a medical endoscope 2 comprising the following steps:
a) having a medical endoscope 2 including an operating handle 3, an insertion tube 4, and optionally a power cable 5,
b) having a tray 6 provided with an inner face 6ᵢ having a peripheral area 6ₚ, said tray 6 comprising on its inner face 6ᵢ a housing 8 intended to receive a medical endoscope 2, the housing 8 being composed of a cavity 9 intended to receive the operating handle 3, a rectilinear groove 10 intended to receive the insertion tube 4, and optionally a gutter 11 intended to receive the power cable 5, the groove 10 having a section greater than the section of the insertion tube 4 to allow its free insertion, said tray 6 comprising protuberances 12ₐ projecting from its inner face 6ᵢ and arranged on either side of the groove 10,
c) providing a waterproof and heat-sealable sheet 7,
d) placing the medical endoscope 2 in the housing 8,
e) contacting the heat-sealable sheet 7 with the peripheral area 6ₚ and with the protuberances 12ₐ, heating to a sealing temperature, applying a peripheral sealing pressure on the peripheral area 6ₚ allowing hermetic sealing of the sheet 7 on the peripheral area 6ₚ to obtain a pull-out force, and applying a central sealing pressure on the protuberances 12ₐ allowing the sheet 7 to be fixed on the protuberances 12ₐ to obtain a pull-out force, the peripheral sealing pressure and the central sealing pressure being such that the pull-out force between the sheet 7 and the protuberances 12ₐ is at least 30% less than the pull-out force between the sheet 7 and the peripheral area 6ₚ.

In step b), the tray 6 is as described above for the packaging 1 according to the present disclosure.

In step c), the sheet 7 is as described above for the packaging 1 according to the present disclosure.

In step e), the heating can be carried out by conduction, IR radiation, laser beam, or ultrasound using a sonotrode for example. The sealing temperature is defined as the temperature allowing heat-sealing between the tray 6 and the sheet 7. This temperature depends on the materials used for producing the tray 6 and the sheet 7, and can be determined by the person skilled in the art.

In step e), the pressure is applied using a sonotrode for example.

In step e), a peripheral sealing pressure is applied to the sheet 7 and the peripheral area 6ₚ of the tray 6, preferably concomitantly with the heating, which allows hermetic sealing around the entire periphery of the packaging 1, between the sheet 7 and the peripheral area 6ₚ of the tray 6. This heating combined with the application of the peripheral pressure allows heat-sealing between the peripheral area 6ₚ of the tray 6 and the sheet 7.

As described above, the tray 6 may include at least one protuberance 12_{b} bordering the gutter 11 and/or at least one protuberance 12_{c} arranged in the gutter 11. According to this embodiment, the central sealing pressure is also applied to the protuberances 12_{b} bordering the gutter 11 and/or the protuberances 12_{c} arranged in the gutter 11, to allow the sheet 7 to be fixed on all the protuberances 12ₐ, 12_{b}, 12_{c} and to obtain a pull-out force. According to this embodiment, the peripheral sealing pressure and the central sealing pressure are such that the pull-out force between the sheet 7 and the protuberances 12ₐ, 12_{b}, 12_{c} is at least 30% less than the pull-out force between the sheet 7 and the peripheral area 6ₚ.

In step e), a central sealing pressure is applied to the sheet 7 and the protuberances 12ₐ, 12_{b}, 12_{c} projecting from the tray 6, preferably concomitantly with the heating, which allows to fix the sheet 7 on the upper face 12ₛ of the protuberances 12. Thus, the sheet 7 is fixed to the protuberances 12ₐ, 12_{b}, 12_{c}, and more precisely to the upper face 12ₛ of the protuberances 12ₐ, 12_{b}, 12_{c}. In the context of the present disclosure, the pull-out force between the sheet 7 and the peripheral area 6ₚ of the tray 6 is at least 30% greater than the pull-out force between the sheet 7 and the protuberances 12ₐ, 12_{b}, 12_{c}. For this purpose, the central sealing pressure is lower than the peripheral sealing pressure. According to a first embodiment, in step b), the peripheral area 6ₚ and the protuberances 12ₐ, 12_{b}, 12_{c} project at the same height relative to the inner surface 6ᵢ of the tray 6. According to this embodiment, the pressure applied in step e) at the peripheral area 6ₚ is then higher than the pressure applied at the protuberances 12ₐ, 12_{b}, 12_{c}. According to a second embodiment, in step b), the protuberances 12ₐ, 12_{b}, 12_{c} project at a height less than that of the peripheral area 6ₚ relative to the inner surface 6ᵢ of the tray 6. According to this embodiment, in step e), the pressure applied at the peripheral area 6ₚ can be identical to that applied at the protuberances 12ₐ, 12_{b}, 12_{c}.

1. A packaging of a medical endoscope comprising an operating handle and an insertion tube, said packaging comprising:
a tray provided with an inner face having a peripheral area, said tray comprising on its inner face a housing receiving the medical endoscope, the housing comprising a cavity receiving the operating handle, and a rectilinear groove receiving the insertion tube, the groove having a section greater than the section of the insertion tube to allow its free insertion,
a sheet permeable to gases and beta and gamma radiation, impermeable to microbes, sealed continuously and hermetically to the peripheral area of the inner face of the tray with a pull-out force,
characterized in that the tray comprises protuberances projecting from its inner face and arranged on either side of the groove,
in that the sheet is fixed to the protuberances with a pull-out force,
and in that the ratio between the pull-out force between the sheet and the protuberances and the pull-out force between the sheet and the peripheral area at isosurface is less than or equal to 0.7.

2. The packaging according to claim 1, wherein the protuberances are positioned at the border of the groove.

3. The packaging according to claim 1, wherein the protuberances (12a) are distributed along the groove.

4. The packaging according to claim 1, wherein the protuberances are arranged alternately on one side or the other of the groove.

5. The packaging according to claim 1, wherein the housing comprises a gutter receiving a power cable of the medical endoscope.

6. The packaging according to the preceding claim, according to which at least one protuberance is positioned at the border of the gutter.

7. The packaging according to claim 5, wherein at least one protuberance is present in the gutter in order to wind the power cable.

8. The packaging according to claim 1, wherein the protuberances are studs or ribs.

9. The packaging according to claim 1, comprising two stop lines arranged in the parts of the peripheral area located on either side of the groove.

10. The packaging according to claim 1, wherein the housing includes clips for holding the operating handle in position.

11. The packaging according to claim 1, according to which the housing comprises a cell receiving a connector of the power cable of the medical endoscope.

12. The packaging according to claim 1, comprising a hanging slot in the peripheral area.

13. The packaging according to claim 1 further comprising an adhesive surface on the inner face of the tray, covered with a peelable film.

14. A method for packaging a medical endoscope comprising the following steps:
a) having a medical endoscope including an operating handle, an insertion tube, and optionally a power cable,
b) having a tray provided with an inner face having a peripheral area, said tray comprising on its inner face a housing intended to receive a medical endoscope, the housing being composed of a cavity intended to receive the operating handle, a rectilinear groove intended to receive the insertion tube, and optionally a gutter intended to receive the power cable, the groove having a section greater than the section of the insertion tube to allow its free insertion, said tray comprising protuberances projecting from its inner face and arranged on either side of the groove,
c) having a sheet permeable to gases and beta and gamma radiation, impermeable to microbes and heat-sealable,
d) placing the medical endoscope in the housing,
e) contacting the heat-sealable sheet with the peripheral area and with the protuberances, heating to a sealing temperature, applying a peripheral sealing pressure on the peripheral area allowing hermetic sealing of the sheet on the peripheral area to obtain a pull-out force, and applying a central sealing pressure on the protuberances allowing the sheet to be fixed on the protuberances to obtain a pull-out force, the peripheral sealing pressure and the central sealing pressure being such that the pull-out force between the sheet and the protuberances is at least 30% less than the pull-out force between the sheet and the peripheral area.

15. The method according to claim 14, according to which, in step b), the peripheral area and the protuberances project at the same height relative to the inner surface of the tray.

16. The method according to claim 14, according to which, in step b), the protuberances project at a height less than that of the peripheral area relative to the inner surface of the tray.
